(19) United States
(12) Patent Application Publication
(10) Pub. No.: US 2022/0160243 A1
(43) Pub. Date: May 26, 2022

(54) PULSE DETERMINATION APPARATUS, STRESS DETERMINATION APPARATUS, PULSE DETERMINATION METHOD, AND COMPUTER-READABLE RECORDING MEDIUM

(71) Applicant: NEC Corporation, Minato-ku, Tokyo (JP)

(72) Inventors: Yoshiki NAKASHIMA, Tokyo (JP); Yoshifumi ONISHI, Tokyo (JP)

(73) Assignee: NEC Corporation, Minato-ku, Tokyo (JP)

(21) Appl. No.: 17/437,955

(22) PCT Filed: Feb. 7, 2020
(86) PCT No.: PCT/JP2020/004949

§ 371 Date: Sep. 10, 2021

(30) Foreign Application Priority Data
Mar. 20, 2019 (JP) 2019-053758

(51) Int. Cl. A61B 5/0245; A61B 5/16

(52) U.S. Cl. CPC A61B 5/0245; A61B 5/16

(57) ABSTRACT

A pulse determination apparatus determines a pulse rate of a person to be measured, determines whether the person to be measured is in a first state in which a pacemaker attached to the person to be measured is operating, a second state in which the pacemaker attached to the person to be measured is not operating, or a third state in which the person to be measured does not wear the pacemaker, based on variation of the pulse rate, and determines whether the pulse of the person to be measured is normal. The pulse determination apparatus does not make a determination, when it is determined that the person to be measured is in the first state, and the pulse determination apparatus makes the determination based on the pulse rate, when it is determined that the person to be measured is in the second state or the third state.

### TECHNICAL FIELD

The invention relates to a pulse determination apparatus for determining whether a pulse is normal or arrhythmia, a stress determination apparatus using the pulse determination apparatus, and a pulse determination method, and further relates to a computer-readable recording medium in which a program for realizing them is recorded.

### BACKGROUND ART

In recent years, it has become a problem that sympathetic nerve is too active due to excessive stress and the like, and the mind and body are tired to impair the health. Therefore, a technique has been proposed in which a wearable terminal is attached to a person to be measured, biological information of the person to be measured is acquired from the wearable terminal, and stress of the person to be measured is monitored.

Patent Document 1 discloses a tension state estimation apparatus using biological information. Patent Document 1 discloses a method for correctly estimating a tension state even when noise is mixed in the measured biological information. According to Patent Document 1, it is possible to use the tension state estimation apparatus even in a daily scene where the noise frequently occurs.

### LIST OF RELATED ART DOCUMENTS

### Patent Document

Patent Document 1: Japanese Patent Laid-Open Publication No. H09-220208

### SUMMARY OF INVENTION

### Problems to be Solved by the Invention

Incidentally, when monitoring the stress of the person to be measured, Heart Rate Variability, which is known to reflect function of autonomic nerve, is particularly used. However, when the person to be measured wears a pacemaker for treatment of arrhythmia, and the pacemaker operates, the pulse of the person to be measured is constant and does not vary. Further, when the person to be measured has arrhythmia, the Heart Rate Variability is irregular. In such a case, an erroneous stress determination may be made. This problem cannot be solved by Patent Document 1.

An example object of the invention is to provide a pulse determination apparatus capable of acquiring a normal pulse that can be used for health management of the person to be measured, a stress determination apparatus, a pulse determination method, and a computer-readable recording medium.

### Means for Solving the Problems

In order to achieve the above object, a pulse determination apparatus in an example aspect of the invention is a pulse determination apparatus that determines a pulse of a person to be measured, comprising:
a pulse rate determination unit configured to determine a pulse rate of the person to be measured;
a state determination unit configured to determine whether the person to be measured is in a first state in which a pacemaker attached to the person to be measured is operating, a second state in which the pacemaker attached to the person to be measured is not operating, or a third state in which the person to be measured does not wear the pacemaker, based on variation of the pulse rate; and
a pulse determination unit configured to determine whether the pulse of the person to be measured is normal, in which
the pulse determination unit does not make a determination, when the state determination unit determines that the person to be measured is in the first state, and
the pulse determination unit makes the determination based on the pulse rate, when the state determination unit determines that the person to be measured is in the second state or the third state.

Further, in order to achieve the above object, a stress determination apparatus in an example aspect of the invention is a stress determination apparatus that determines stress of a person to be measured, comprising:
a biological information acquisition unit configured to acquire biological information of the person to be measured;
a pulse rate determination unit configured to determine a pulse rate of the person to be measured from the biological information acquired by the biological information acquisition unit;
a state determination unit configured to determine whether the person to be measured is in a first state in which a pacemaker attached to the person to be measured is operating, a second state in which the pacemaker attached to the person to be measured is not operating, or a third state in which the person to be measured does not wear the pacemaker, based on variation of the pulse rate;
a pulse determination unit configured to determine whether a pulse of the person to be measured is normal; and
a stress determination unit configured to determine the stress of the person to be measured,
wherein the pulse determination unit does not make a determination, when the state determination unit determines that the person to be measured is in the first state,
the pulse determination unit makes the determination based on the pulse rate, when the state determination unit determines that the person to be measured is in the second state or the third state,
the stress determination unit determines the stress of the person to be measured by using the pulse rate, when the pulse determination unit determines that the pulse of the person to be measured is normal, and
the stress determination unit determines the stress of the person to be measured by using the biological information other than the pulse rate, when the pulse determination unit determines that the pulse of the person to be measured is not normal, or when the state determination unit determines that the person to be measured is in the first state.

Further, in order to achieve the above object, a pulse determination method in an example aspect of the invention is a pulse determination method for determining a pulse of a person to be measured, comprising:
a step of determining a pulse rate of the person to be measured;
a step of determining whether the person to be measured is in a first state in which a pacemaker attached to the person to be measured is operating, a second state in which the pacemaker attached to the person to be measured is not operating, or a third state in which the person to be measured does not wear the pacemaker, based on variation of the pulse rate; and
a step of determining whether the pulse of the person to be measured is normal,
wherein in the step of determining whether the pulse is normal,
a determination is not made, when the person to be measured is determined to be in the first state, and
the determination is made based on the pulse rate, when it is determined to be in the second state or the third state.

Further, in order to achieve the above object, a computer-readable recording medium in an example aspect of the invention is a computer-readable recording medium that records a program that causes a computer to determine a pulse of a person to be measured,
wherein the computer-readable recording medium records a program including instructions that cause the computer to perform:
a step of determining whether the person to be measured is in a first state in which a pacemaker attached to the person to be measured is operating, a second state in which the pacemaker attached to the person to be measured is not operating, or a third state in which the person to be measured does not wear the pacemaker, based on determined variation of a pulse rate of the person to be measured; and
a step of determining whether the pulse of the person to be measured is normal,
wherein in the step of determining whether the pulse is normal,
a determination is not made, when the person to be measured is determined to be in the first state, and
the determination is made based on the pulse rate, when it is determined to be in the second state or the third state.

### Advantageous Effects of the Invention

According to the invention, it is determined whether the pulse is normal or arrhythmia when the pacemaker is not operating. As a result of the determination, the pulse rate when the pulse is normal can be used for the health management of the person to be measured, and the health management of the person to be measured can be performed accurately.

### BRIEF DESCRIPTION OF THE DRAWINGS

FIG. 1 is a block diagram illustrating a schematic configuration of a pulse determination apparatus according to an example embodiment of the invention.

FIG. 2 is a block diagram illustrating a specific configuration of a stress determination apparatus according to the example embodiment of the invention. FIG. 3 is a diagram illustrating waveforms of time-series data of pulse rate during pacemaker operation and non-operation.

FIG. 4 is a diagram illustrating waveforms of time-series data of pulse rate in the case where the pulse is normal and the case where the pulse is arrhythmia.

FIG. 5 is a flowchart illustrating an operation of the stress determination apparatus.

FIG. 6 is a flowchart illustrating an operation of a pulse determination process.

FIG. 7 is a block diagram illustrating an example of a computer that realizes the pulse determination apparatus and the stress determination apparatus according to the example embodiment of the invention.

### EXAMPLE EMBODIMENT

### Example Embodiment

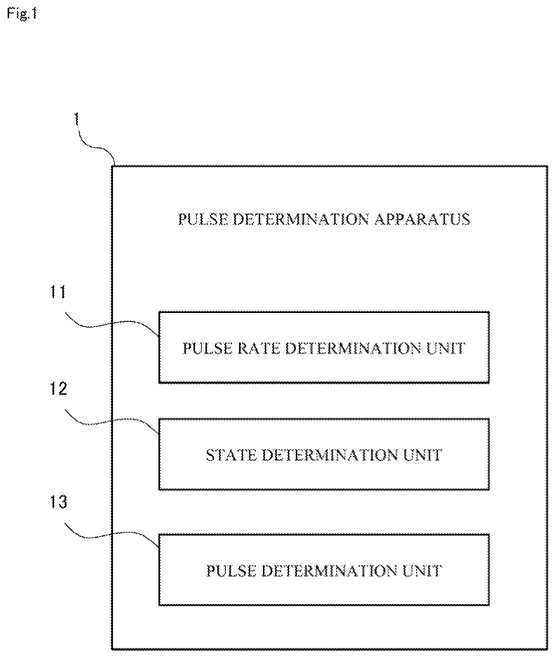

Hereinafter, a pulse determination apparatus, a stress determination apparatus, a pulse determination method, and a program according to an example embodiment of the invention will be described with reference to FIGS. 1 to 7.

### Apparatus Configuration

FIG. 1 is a block diagram illustrating a schematic configuration of a pulse determination apparatus 1 according to the example embodiment of the invention.

The pulse determination apparatus 1 is an apparatus that determines a pulse of a person to be measured.

The pulse determination apparatus 1 includes a pulse rate determination unit 11, a state determination unit 12, and a pulse determination unit 13.

The pulse rate determination unit 11 determines a pulse rate [bpm] of the person to be measured. Specifically, the pulse rate determination unit 11 determines time-series data of the pulse rate of the person to be measured. In the example embodiment, the pulse rate determination unit 11 acquires the time-series data of the pulse rate from a wearable terminal.

The state determination unit 12 determines whether the person to be measured is in a first state, a second state, or a third state based on the time-series data of the pulse rate. The first state is a state in which a pacemaker attached to the person to be measured is operating. The second state is a state in which the pacemaker attached to the person to be measured is not operating. The third state is a state in which the person to be measured does not wear the pacemaker.

The pulse determination unit 13 determines whether the pulse of the person to be measured is normal or arrhythmia (hereinafter, referred to as pulse determination). When the state determination unit 12 determines that the person to be measured is in the first state, the pulse determination unit 13 does not make the pulse determination. When the state determination unit 12 determines that the person to be measured is in the second state or the third state, the pulse determination unit 13 makes the pulse determination for the time-series data of the pulse rate.

When the pulse of the person to be measured wearing the pacemaker is disturbed, the pacemaker operates to stabilize the pulse of the person to be measured. In this case, since the pulse of the person to be measured is constant, the pulse determination unit 13 determines that the pulse of the person to be measured is normal. That is, the pulse determination unit 13 always determines that the pulse is normal even if the pulse determination is made during pacemaker operation. Therefore, the pulse determination apparatus 1 of the example embodiment makes the pulse determination only when the person to be measured does not wear the pacemaker, or when the person to be measured wears the pacemaker but the pacemaker is not operating. As a result, it is possible to accurately determine whether the pulse of the person to be measured is normal.

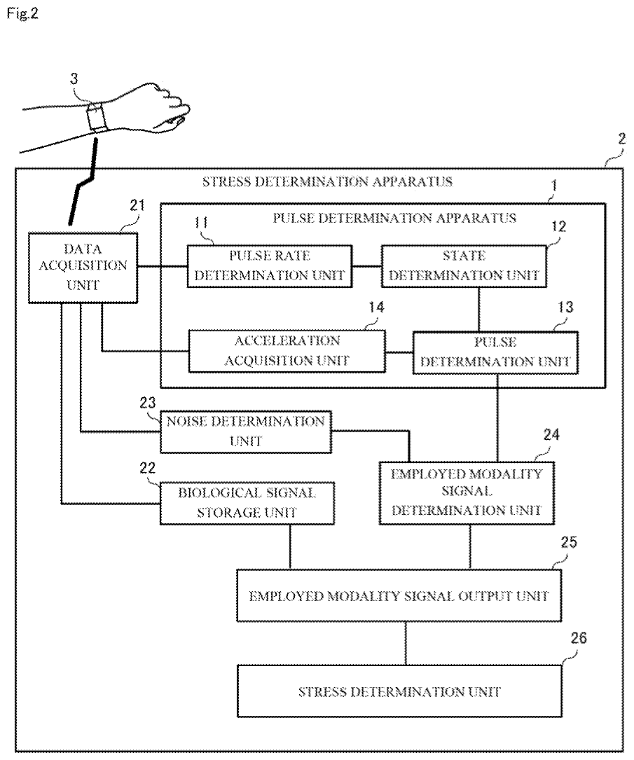

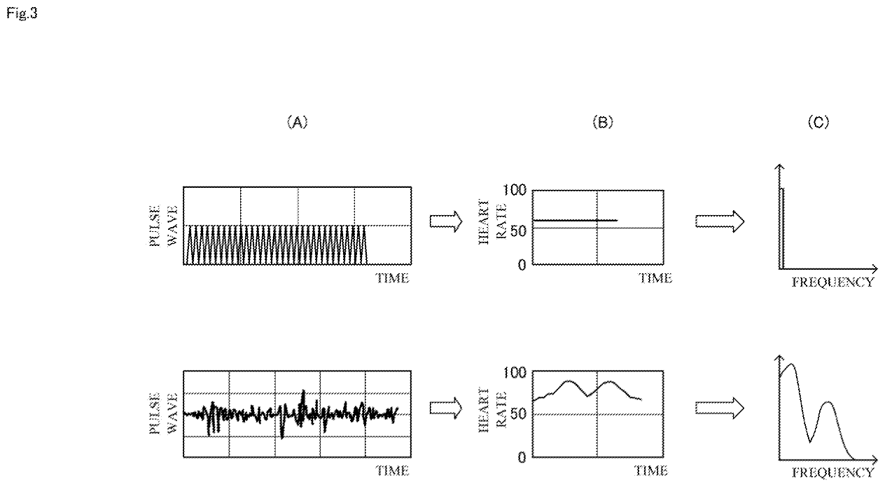

Subsequently, the configuration of the pulse determination apparatus 1 of the example embodiment will be specifically described with reference to FIGS. 2 to 7. The pulse determination apparatus 1 is used as a partial configuration of the stress determination apparatus that determines stress of the person to be measured. Hereinafter, the stress determination apparatus will be specifically described.

FIG. 2 is a block diagram illustrating a specific configuration of a stress determination apparatus 2 according to the example embodiment of the invention.

The stress determination apparatus 2 includes the pulse determination apparatus 1, a data acquisition unit 21, a biological signal storage unit 22, a noise determination unit 23, an employed modality signal determination unit 24, an employed modality signal output unit 25, and a stress determination unit 26.

The stress determination apparatus 2 is constructed by a computer. In the example embodiment, the stress determination apparatus 2 can perform wired or wireless data communication with a part of a body of the person to be measured, for example, a wearable terminal 3 worn on an arm. Note that the wearable terminal 3 may perform data communication with a mobile device terminal (for example, a smartphone) owned by the person to be measured, and the stress determination apparatus 2 and the wearable terminal 3 may perform data communication via the mobile device terminal.

The wearable terminal 3 measures the pulse rate of the person to be measured. The wearable terminal 3 transmits a biological signal and an acceleration signal. The wearable terminal 3 has an acceleration sensor. The acceleration signal is an output signal of the acceleration sensor. The biological signal is a signal on biological information of the person to be measured. The biological information is information on the pulse rate, an amount of sweating, and a skin temperature of the person to be measured.

The wearable terminal 3 measures the pulse rate from a pulse wave of the person to be measured detected by a pulse wave sensor. Further, the wearable terminal 3 measures the amount of sweating of the person to be measured from a change in electrical resistance value of a skin of the person to be measured detected by a humidity sensor. Furthermore, the wearable terminal 3 measures the skin temperature of the person to be measured from intensity of infrared rays emitted from the skin of the person to be measured detected by a thermosensor. The wearable terminal 3 generates time-series data for each of the pulse rate, the amount of sweating, and the skin temperature, and transmits the time-series data as the biological signal to the stress determination apparatus 2.

The data acquisition unit 21 acquires the biological information of the person to be measured by acquiring the biological signal transmitted from the wearable terminal 3. The data acquisition unit 21 is a “biological information acquisition unit”. Further, the data acquisition unit 21 also acquires the acceleration signal transmitted from the wearable terminal 3.

The biological signal storage unit 22 stores the biological signal. The biological signal storage unit 22 stores the biological signal each time the data acquisition unit 21 acquires the biological signal.

The noise determination unit 23 determines whether biological signals other than the pulse rate, that is, the biological signals of the amount of sweating and the skin temperature, are used as noise signals among the biological signals acquired by the data acquisition unit 21. The biological signal as the noise signal is not used for stress determination in the stress determination unit 26 described below. Further, the biological signal that has not been determined to be the noise signal is determined to be a normal signal, and is used for the stress determination in the stress determination unit 26 described below. The noise determination unit 23 outputs a determination result to the employed modality signal determination unit 24.

In the case of the biological signal of the skin temperature, if the skin temperature acquired from the wearable terminal 3 is not within an allowable range of a surface skin temperature of a human, for example, 25° C. to 40° C., the noise determination unit 23 determines that the biological signal of the skin temperature is the noise signal. If the skin temperature is within the allowable range, the noise determination unit 23 determines that the biological signal of the skin temperature is the normal signal.

In the case of the biological signal of the amount of sweating, for example, the stress determination apparatus 2 also receives a signal of the electrical resistance value for measuring the amount of sweating from the wearable terminal 3. Then, the noise determination unit 23 performs artifact removal or the like by wavelet transform on the signal, and determines that the biological signal of the amount of sweating is the noise signal if the electrical resistance value is not within a preset threshold range. The noise determination unit 23 determines that the biological signal of the amount of sweating is the normal signal if the electrical resistance value is within the threshold range. The artifact removal by wavelet transform is disclosed, for example, in “W. Chen, N. Jaques, S. Taylor, A. Sano, S. Fedor and R. W. Picard, “Wavelet-based motion artifact removal for electrodermal activity”, 2015 37th Annual International Conference of the IEEE Engineering in Medicine and Biology Society (EMBC), 2015, pp. 6223-6226”.

The pulse determination apparatus 1 makes the pulse determination for the biological signal of the pulse rate. The pulse determination apparatus 1 determines that the biological signal of the pulse rate is the normal signal when the pulse is normal, and determines that the biological signal of the pulse rate is the noise signal when the pulse is arrhythmia. Then, the pulse determination apparatus 1 outputs the determination result to the employed modality signal determination unit 24. The employed modality signal determination unit 24 determines the biological signal determined to be the normal signal by the noise determination unit 23 and the pulse determination apparatus 1 among the biological signals acquired by the data acquisition unit 21, and outputs the biological signal to the employed modality signal determination unit 24.

The employed modality signal output unit 25 acquires the biological signal determined by the employed modality signal determination unit 24 from the biological signal storage unit 22. Then, the employed modality signal output unit 25 outputs the acquired biological signal to the stress determination unit 26.

The stress determination unit 26 makes the stress determination using the biological signal output from the employed modality signal output unit 25. The stress determination made by the stress determination unit 26 can use a well-known method and is not particularly limited.

The pulse rate determination apparatus 1 includes an acceleration acquisition unit 14 in addition to the pulse rate determination unit 11, the state determination unit 12, and the pulse determination unit 13 described above.

The acceleration acquisition unit 14 acquires acceleration of the wearable terminal 3 from the acceleration signal acquired by the data acquisition unit 21. The pulse determination apparatus 1 estimates an exercise state of the person to be measured from the acceleration of the wearable terminal 3.

The pulse rate determination unit 11 acquires the biological signal of the pulse rate among the biological signals acquired by the data acquisition unit 21. Note that in the example embodiment, the stress determination apparatus 2 acquires the pulse rate measured by the wearable terminal 3, but may be configured to receive the pulse wave of the person to be measured from the wearable terminal 3 and calculate the pulse rate from the received pulse wave.

The state determination unit 12 determines whether the person to be measured is in the first state, the second state, or the third state described above from the time-series data of the pulse rate. The determination method by the state determination unit 12 will be described below.

FIG. 3 is a diagram illustrating waveforms of the time-series data of the pulse rate during pacemaker operation and non-operation. The upper figure of FIG. 3 illustrates the time-series data of the pulse rate when the pacemaker is operating, and the lower figure illustrates the time-series data of the pulse rate when the pacemaker is not operating.

(A) of FIG. 3 is the waveform of the time-series data of the pulse wave detected by the pulse wave sensor of the wearable terminal 3. The pulse wave is blood flow in a blood vessel and moves up and down in accordance with the heartbeat. A time width between peaks of the pulse wave detected when the pacemaker is operating is always constant. On the other hand, the time width between the peaks of the pulse wave detected when the pacemaker is not operating varies with time.

(B) of FIG. 3 is the waveform of the time-series data of the pulse rate [bpm] calculated from the pulse wave. The pulse rate is a value recorded every minute by counting the number of times the blood flow goes up and down. For example, if the blood flow goes up and down 60 times in one minute, a heart rate is 60 bpm. When the pacemaker is operating, the pulse rate is always constant. On the other hand, when the pacemaker is not operating, the pulse rate varies with time.

The state determination unit 12 performs frequency analysis on the time-series data of the pulse rate. (C) of FIG. 3 is a waveform representing power spectral density with respect to a frequency obtained by the frequency analysis. The heart rate illustrated in (B) of FIG. 3 is not always constant, increases due to strenuous exercise, and decreases when resting. It is known that there is a certain cycle in heart rate variation. (C) of FIG. 3 illustrates a frequency of this variation in the form of power spectral density. (C) of FIG. 3 indicates which frequency of the heart rate variation is many by monitoring the heart rate over a long period of time. The state determination unit 12 determines whether a peak value of the power spectral density exists in a low frequency region (0.05 to 0.15 Hz) and a high frequency region (0.15 to 0.4 Hz) as a result of the frequency analysis. Sympathetic and parasympathetic activity appears in the low frequency region, and the parasympathetic activity appears in the high frequency region. In the case of the pulse wave kept constant by an operation of the pacemaker, there is no peak value in the low frequency region and the high frequency region.

Therefore, if there are no peak value in the low frequency region and the high frequency region, the state determination unit 12 determines that the pulse rate measured by the wearable terminal 3 is the pulse rate when the pacemaker is operating, and determines that the person to be measured is in the first state. On the other hand, when a peak exists in each frequency region, the state determination unit 12 determines that the pulse rate measured by the wearable terminal 3 is the pulse rate when the pacemaker is not operating, and determines that it is in the second state or the third state.

Note that the state determination unit 12 may determine whether the person to be measured is in the first state, the second state, or the third state from the time-series data of the pulse rate illustrated in (B) of FIG. 3 before performing the frequency analysis. For example, the state determination unit 12 determines that the person to be measured is in the first state when the pulse rate is always constant, or when the pulse rate increases or decreases at a constant rate. When the pulse rate variates irregularly in time series, the state determination unit 12 determines that it is in the second state or the third state.

The pulse determination unit 13 makes the pulse determination of whether the pulse of the person to be measured is normal or arrhythmia according to the determination result of the state determination unit 12. Arrhythmia is bradycardia or tachycardia. The pulse determination unit 13 does not make the pulse determination when the state determination unit 12 determines that the person to be measured is in the first state, that is, when the person to be measured wears the pacemaker and the pacemaker is operating. As described above, when the pacemaker worn by the person to be measured is operating, the pulse rate of the person to be measured is kept constant. There is no variation in the pulse rate at this time, and even if it is determined whether the pulse rate is normal or arrhythmic, the pulse determination unit 13 always determines that the pulse is normal. Therefore, the pulse determination result cannot be obtained accurately.

Therefore, when the state determination unit 12 determines that it is in the second state or the third state, that is, when the person to be measured does not wear the pacemaker, or when the person to be measured wears the pacemaker but the pacemaker is not operating, the pulse determination unit 13 makes the pulse determination. The pulse determination unit 13 determines that the biological signal of the pulse rate is the normal signal when the pulse is normal as a result of the pulse determination. When the pulse is arrhythmia, the pulse determination unit 13 determines that the biological signal of the pulse rate is the noise signal. The pulse determination unit 13 outputs the determination result to the employed modality signal determination unit 24. Note that the pulse determination method by the pulse determination unit 13 will be described below.

Further, the pulse determination unit 13 makes the pulse determination in consideration of the acceleration of the wearable terminal 3. When the person to be measured is exercising, since the pulse rate of the person to be measured increases, the pulse when the person to be measured is exercising has a greater variation in the time width between the peaks than that of the normal pulse. Therefore, the pulse determination unit 13 may determine that the pulse is arrhythmia even if the pulse is normal. Therefore, the pulse determination unit 13 corrects by the acceleration the time-series data of the pulse rate for which the pulse determination is made, and converts the time-series data into a value that takes into account the exercise state of the person to be measured. Examples of this conversion method include methods described in the document “Comparing VO2max determined by using the relation between heart rate and accelerometry with submaximal estimated VO2max”, “O. TIKKANEN, EMG, Heart Rate, and Accelerometer as Estimators of Energy Expenditure in Locomotion”, or the like.

A function of the pulse determination unit 13 will be described below.

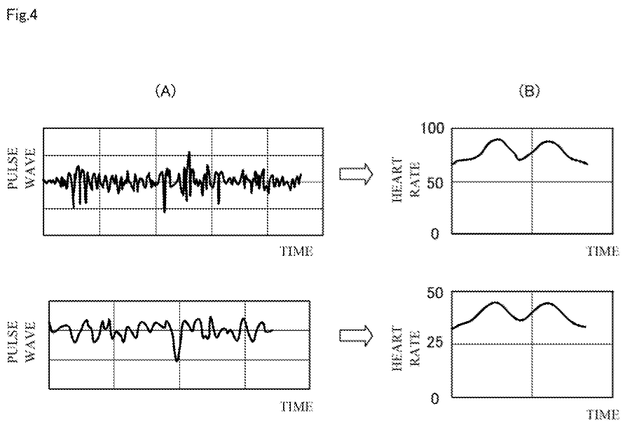

FIG. 4 is a diagram illustrating the waveforms of the time-series data of the pulse rate in the case where the pulse is normal and the case where the pulse is arrhythmia. The upper figure of FIG. 4 illustrates the time-series data of the pulse rate when the pulse is normal, and the lower figure illustrates the time-series data of the pulse rate when the pulse is arrhythmia (bradycardia in FIG. 4). (A) of FIG. 4 illustrates the time-series data of the pulse wave. (B) of FIG. 4 is the time-series data of the pulse rate [bpm] calculated from the pulse wave.

The pulse determination unit 13 performs power spectrum analysis on the time-series data of the pulse rate after correction by acceleration, and determines that the pulse is bradycardia, that is, the pulse is arrhythmia when a frequency of a certain threshold value (for example, 50 bpm) or less continues. Further, the pulse determination unit 13 determines that the pulse is tachycardia when a frequency of a certain threshold value (for example, 100 bpm) or more continues.

Further, the pulse determination unit 13 makes the pulse determination in consideration of tendency of athlete's heart. A person with the “athlete's heart” is, for example, a person who regularly performs intense aerobic exercise, and the person's pulse tends to be bradycardia. When the person to be measured has the athlete's heart, the pulse is normal even if the pulse rate is 50 bpm or less. Therefore, the pulse determination unit 13 determines whether the person to be measured has the athlete's heart by using a method described, for example, in the document “Vanessa Pereira da Silva et al., Heart Rate Variability Indexes as a Marker of Chronic Adaptation in Athletes A Systematic Review” so that the pulse of the person to be measured with the athlete's heart is not determined to be bradycardia even though the pulse is normal.

### Operation of Apparatus

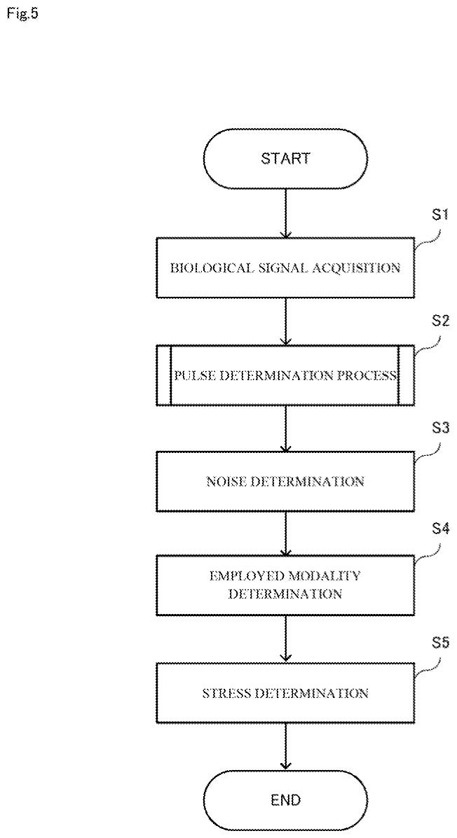

Next, an operation of the stress determination apparatus 2 according to the example embodiment of the invention will be described with reference to FIG. 5. FIG. 5 is a flowchart illustrating the operation of the stress determination apparatus 2. In the following description, FIG. 2 will be referred to as appropriate. Further, in the example embodiment, the pulse determination method is performed by operating the stress determination apparatus 2. Therefore, the description of the pulse determination method in the example embodiment is replaced with the following description of the operation of the stress determination apparatus 2.

First, as a premise, a communication connection is made between the stress determination apparatus 2 and the wearable terminal 3 so that the data communication can be made. It is assumed that the wearable terminal 3 measures the pulse rate, the skin temperature, and the amount of sweating of the person to be measured, and transmits the time-series data of them as the biological signal to the stress determination apparatus 2. Further, it is assumed that the wearable terminal 3 transmits the acceleration signal to the stress determination apparatus 2 when it detects the acceleration.

The data acquisition unit 21 of the stress determination apparatus 2 acquires the biological signal transmitted from the wearable terminal 3 (S1). Subsequently, the pulse determination apparatus 1 performs a pulse determination process on the biological signal of the pulse rate (S2).

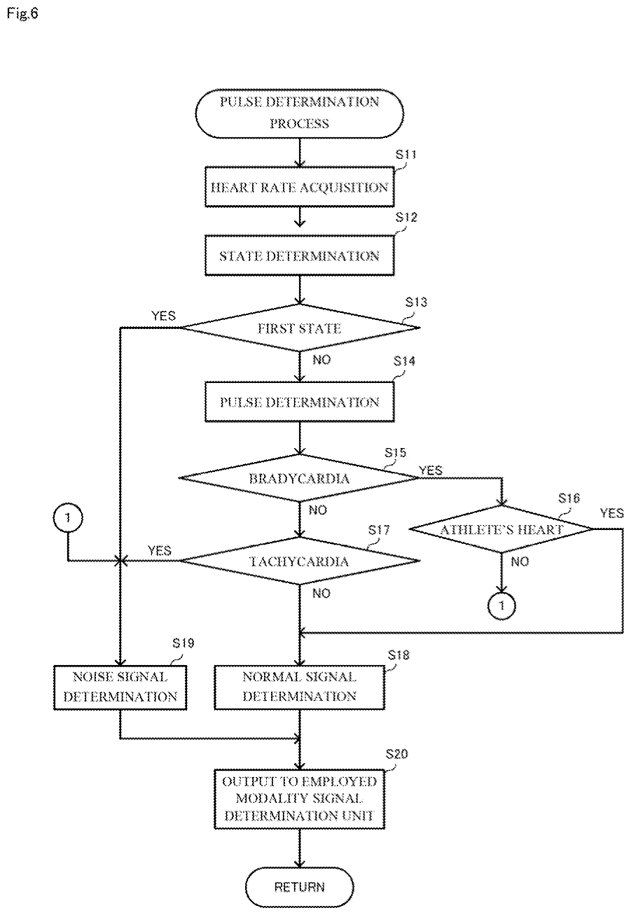

FIG. 6 is a flowchart illustrating an operation of the pulse determination process.

The pulse rate determination unit 11 of the pulse determination apparatus 1 acquires the biological signal of the pulse rate from the biological signal acquired by the data acquisition unit 21 (S11). Subsequently, the state determination unit 12 determines whether the person to be measured is in the first state, the second state, or the third state from the time-series data of the pulse rate (S12). In the state determination, the frequency analysis is performed on the time-series data of the pulse rate, and it is determined whether the peak value of the power spectral density exists in the low frequency region (0.05 to 0.15 Hz) and the high frequency region (0.15 to 0.4 Hz). The state determination unit 12 determines that the person to be measured is in the first state if the peak value does not exist, and determines that it is in the second state or the third state if the peak value exists.

As a result of the state determination, the state determination unit 12 determines whether the person to be measured is in the first state (S13). When the person to be measured is determined to be in the first state (S13: YES), it is determined that the person to be measured wears the pacemaker, and the pacemaker is operating, and the pulse determination unit 13 determines that the biological signal of the pulse rate is the noise signal (S19). In this case, the stress determination unit 26 makes the stress determination without using the biological signal of the pulse rate.

When it is determined that the person to be measured is not in the first state (S13: NO), that is, when the state determination unit 12 determines that it is in the second state or the third state, the pulse determination unit 13 makes the pulse determination (S14). In the pulse determination, as described above, the power spectrum analysis is performed on the time-series data of the pulse rate, and when the frequency of the certain threshold value (for example, 50 bpm) or less continues, the pulse is determined to be bradycardia, and when the frequency of the certain threshold value (for example, 100 bpm) or more continues, the pulse is determined to be tachycardia.

The pulse determination unit 13 determines whether the pulse is bradycardia (S15). When it is determined that the pulse is bradycardia (S15: YES), the pulse determination unit 13 determines whether it is the athlete's heart (S16). When it is not the athlete's heart (S16: NO), the pulse determination unit 13 determines that the biological signal of the pulse rate is the noise signal (S19). When it is the athlete's heart (S16: YES), the pulse determination unit 13 determines that the biological signal of the pulse rate is the normal signal (S18). In this case, the stress determination unit 26 makes the stress determination using the biological signal of the pulse rate.

If the pulse is not bradycardia (S15: NO), the pulse determination unit 13 determines whether the pulse is tachycardia (S17). When the pulse is tachycardia (S17: YES), the pulse determination unit 13 determines that the biological signal of the pulse rate is the noise signal (S19). When the pulse is not tachycardia (S17: NO), the pulse determination unit 13 determines that the biological signal of the pulse rate is the normal signal (S18). The pulse determination unit 13 outputs the determination results in S18 and S19 to the employed modality signal determination unit 24 (S20).

Return to FIG. 5. The noise determination unit 23 of the stress determination apparatus 2 determines whether the biological signal of the skin temperature and the amount of sweating is the normal signal or the noise signal (S3). Subsequently, the employed modality signal determination unit 24 determines the biological signal from the determination results output from the noise determination unit 23 and the pulse determination unit 13, and outputs the biological signal to the employed modality signal output unit 25 (S4). The employed modality signal output unit 25 acquires the biological signal from the biological signal storage unit 22, and the stress determination unit 26 makes the stress determination using the acquired biological signal (S5).

As described above, the pulse determination apparatus 1 provided in the stress determination apparatus 2 makes the pulse determination when the person to be measured does not wear the pacemaker, or only when the pacemaker is not operating even if the person to be measured wears the pacemaker. Further, the stress determination apparatus 2 does not use the pulse determined to be arrhythmia as the result of the pulse determination by the pulse determination apparatus 1 for the stress determination, but makes the stress determination using only the pulse determined to be normal. In this way, when the stress determination apparatus 2 makes the stress determination, personal information such as whether the person to be measured wears the pacemaker and whether the person to be measured suffers from arrhythmia is not required in advance, and health management of the person to be measured can be performed accurately.

### Program

It suffices for the program according to the example embodiment of the invention to be a program that causes the computer to execute steps illustrated in FIGS. 5 and 6. By installing this program in the computer and executing it, the pulse determination apparatus, the stress determination apparatus, and the pulse determination method according to the example embodiment can be realized. In this case, a processor of the computer functions and performs processing as the pulse rate determination unit 11, the state determination unit 12, and the pulse determination unit 13. Further, the processor of the computer functions and performs processing as the data acquisition unit 21, the biological signal storage unit 22, the noise determination unit 23, the employed modality signal determination unit 24, the employed modality signal output unit 25, and the stress determination unit 26.

Further, the program in the example embodiment may be executed by a computer system constructed by a plurality of computers. In this case, for example, each computer may function as any of the pulse rate determination unit 11, the state determination unit 12, the pulse determination unit 13, the data acquisition unit 21, the biological signal storage unit 22, the noise determination unit 23, the employed modality signal determination unit 24, the employed modality signal output unit 25 and the stress determination unit 26.

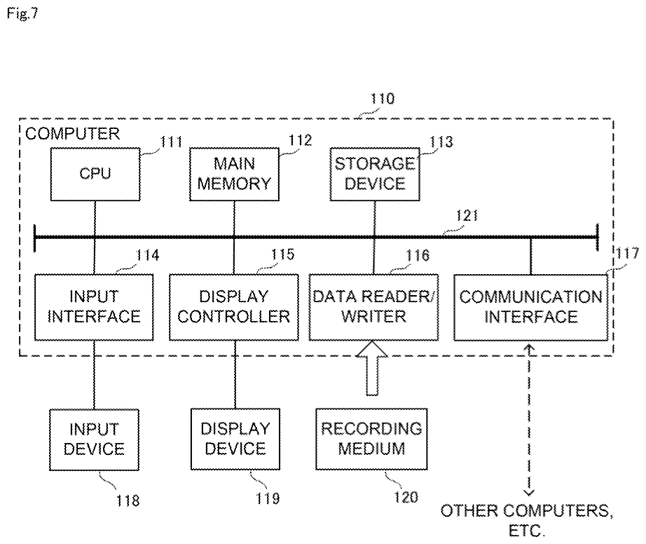

Here, the computer that realizes the pulse determination apparatus 1 and the stress determination apparatus 2 by executing the program in the example embodiment will be described with reference to FIG. 7. FIG. 7 is a block diagram illustrating an example of the computer that realizes the pulse determination apparatus 1 and the stress determination apparatus 2 according to the example embodiment of the invention.

As illustrated in FIG. 7, a computer 110 includes a CPU 111, a main memory 112, a storage device 113, an input interface 114, a display controller 115, a data reader/writer 116, and a communication interface 117. These components are connected to each other via a bus 121 so as to be capable of data communication with one another. Note that the computer 110 may include a graphics processing unit (GPU) or a field-programmable gate array (an FPGA) in addition to the CPU 111 or in place of the CPU 111.

The CPU 111 expands the program (codes) in the example embodiment which is stored in the storage device 113 into the main memory 112 and performs various computations by executing these codes in a predetermined order. The main memory 112 is typically a volatile storage device such as a dynamic random access memory (DRAM). Further, the program in the example embodiment is provided in a state of being stored in a computer-readable recording medium 120. The program in the example embodiment may be distributed on the Internet connected via the communication interface 117.

Further, specific examples of the storage device 113 include a semiconductor storage device such as a flash memory in addition to a hard disk drive. The input interface 114 mediates data transmission between the CPU 111 and an input device 118 such as a keyboard and a mouse. The display controller 115 is connected to a display device 119 and controls display on the display device 119.

The data reader/writer 116 mediates the data transmission between the CPU 111 and the recording medium 120, and executes the reading of the program from the recording medium 120, and the writing of results of processing in the computer 110 to the recording medium 120. The communication interface 117 mediates the data transmission between the CPU 111 and other computers.

Further, specific examples of the recording medium 120 include a general-purpose semiconductor storage device such as a compact flash (CF) (registered trademark) and a secure digital (SD), a magnetic recording medium such as a flexible disk, or an optical recording medium such as a compact disk read only memory (CD-ROM).

The pulse determination apparatus 1 and the stress determination apparatus 2 in the example embodiment can also be realized by using pieces of hardware corresponding to respective units, rather than using a computer in which the program is installed. Further, the pulse determination apparatus 1 and the stress determination apparatus 2 may be partially realized by using a program and the rest may be realized by using hardware.

While a part of or the entirety of the above-described example embodiment can be described by (Supplementary note 1) to (Supplementary note 19) described in the following, the invention is not limited to the following descriptions.

### Supplementary Note 1

A pulse determination apparatus that determines a pulse of a person to be measured, comprising:
a pulse rate determination unit configured to determine a pulse rate of the person to be measured;
a state determination unit configured to determine whether the person to be measured is in a first state in which a pacemaker attached to the person to be measured is operating, a second state in which the pacemaker attached to the person to be measured is not operating, or a third state in which the person to be measured does not wear the pacemaker, based on variation of the pulse rate; and
a pulse determination unit configured to determine whether the pulse of the person to be measured is normal, wherein
the pulse determination unit does not make a determination, when the state determination unit determines that the person to be measured is in the first state, and
the pulse determination unit makes the determination based on the pulse rate, when the state determination unit determines that the person to be measured is in the second state or the third state.

### Supplementary Note 2

The pulse determination apparatus according to Supplementary note 1,
wherein the state determination unit determines that the person to be measured is in the first state when the pulse rate is constant, or when the pulse rate increases or decreases at a constant rate.

### Supplementary Note 3

The pulse determination apparatus according to Supplementary note 1,
wherein the state determination unit performs frequency analysis on the pulse rate, and determines that the person to be measured is in the second state or the third state when a peak value exists in a predetermined frequency range.

### Supplementary Note 4

The pulse determination apparatus according to any one of Supplementary notes 1 to 3, further comprising
an acceleration acquisition unit configured to acquire an acceleration of movement of at least one part of the person to be measured,
wherein the pulse determination unit corrects the pulse rate with the acceleration, and determines the pulse of the person to be measured by the corrected pulse rate.

### Supplementary Note 5

The pulse determination apparatus according to any one of Supplementary notes 1 to 4,
wherein the pulse determination unit performs frequency analysis on the pulse rate, determines that the pulse of the person to be measured is bradycardia when a frequency range of a first threshold value or less exists, and determines that the pulse of the person to be measured is tachycardia when a frequency range of a second threshold value or more exists.

### Supplementary Note 6

The pulse determination apparatus according to any one of Supplementary notes 1 to 5,
wherein the pulse rate determination unit determines the pulse rate of the person to be measured from a pulse wave signal of the person to be measured.

### Supplementary Note 7

A stress determination apparatus that determines stress of a person to be measured, comprising:
a biological information acquisition unit configured to acquire biological information of the person to be measured;
a pulse rate determination unit configured to determine a pulse rate of the person to be measured from the biological information acquired by the biological information acquisition unit;
a state determination unit configured to determine whether the person to be measured is in a first state in which a pacemaker attached to the person to be measured is operating, a second state in which the pacemaker attached to the person to be measured is not operating, or a third state in which the person to be measured does not wear the pacemaker, based on variation of the pulse rate;
a pulse determination unit configured to determine whether a pulse of the person to be measured is normal; and
a stress determination unit configured to determine the stress of the person to be measured,
wherein the pulse determination unit does not make a determination, when the state determination unit determines that the person to be measured is in the first state,
the pulse determination unit makes the determination based on the pulse rate, when the state determination unit determines that the person to be measured is in the second state or the third state,
the stress determination unit determines the stress of the person to be measured by using the pulse rate, when the pulse determination unit determines that the pulse of the person to be measured is normal, and
the stress determination unit determines the stress of the person to be measured by using the biological information other than the pulse rate, when the pulse determination unit determines that the pulse of the person to be measured is not normal, or when the state determination unit determines that the person to be measured is in the first state.

### Supplementary Note 8

A pulse determination method for determining a pulse of a person to be measured, comprising:
step of determining a pulse rate of the person to be measured;
a step of determining whether the person to be measured is in a first state in which a pacemaker attached to the person to be measured is operating, a second state in which the pacemaker attached to the person to be measured is not operating, or a third state in which the person to be measured does not wear the pacemaker, based on variation of the pulse rate; and
a step of determining whether the pulse of the person to be measured is normal,
wherein in the step of determining whether the pulse is normal,
a determination is not made, when the person to be measured is determined to be in the first state, and
the determination is made based on the pulse rate, when the person to be measured is determined to be in the second state or the third state.

### Supplementary Note 9

The pulse determination method according to Supplementary note 8,
wherein when determining whether the person to be measured is in the first state, the second state, or the third state,
it is determined that the person to be measured is in the first state when the pulse rate is constant, or when the pulse rate increases or decreases at a constant rate.

### Supplementary note 10

The pulse determination method according to Supplementary note 8,
wherein when determining whether the person to be measured is in the first state, the second state, or the third state,
frequency analysis is performed on the pulse rate, and it is determined that the person to be measured is in the second state or the third state when a peak value exists in a predetermined frequency range.

### Supplementary Note 11

The pulse determination method according to any one of Supplementary notes 8 to 10, further comprising
a step of acquiring an acceleration of movement of at least one part of the person to be measured,
wherein when determining whether the pulse is normal,
the pulse rate is corrected by the acceleration, and the pulse of the person to be measured is determined by the corrected pulse rate.

### Supplementary Note 12

The pulse determination method according to any one of Supplementary notes 8 to 11,
wherein in the step of determining whether the pulse is normal,
frequency analysis is performed on the pulse rate, and it is determined that the pulse of the person to be measured is bradycardia when a frequency range of a first threshold value or less exists, and is determined that the pulse of the person to be measured is tachycardia when a frequency range of a second threshold value or more exists.

### Supplementary Note 13

The pulse determination method according to any one of Supplementary notes 8 to 12,
wherein when determining the pulse rate,
the pulse rate of the person to be measured is determined from a pulse wave signal of the person to be measured.

### Supplementary Note 14

A computer-readable recording medium that records a program that causes a computer to determine a pulse of a person to be measured,
wherein the computer-readable recording medium records a program including instructions that cause the computer to perform the following steps:
a step of determining whether the person to be measured is in a first state in which a pacemaker attached to the person to be measured is operating, a second state in which the pacemaker attached to the person to be measured is not operating, or a third state in which the person to be measured does not wear the pacemaker, based on determined variation of a pulse rate of the person to be measured; and
a step of determining whether the pulse of the person to be measured is normal, and
in the step of determining whether the pulse is normal,
a determination is not made, when the person to be measured is determined to be in the first state, and
the determination is made based on the pulse rate, when the person to be measured is determined to be in the second state or the third state.

### Supplementary Note 15

The computer-readable recording medium according to Supplementary note 14,
wherein in the step of determining whether the person to be measured is in the first state, the second state, or the third state,
it is determined that the person to be measured is in the first state when the pulse rate is constant, or when the pulse rate increases or decreases at a constant rate.

### Supplementary Note 16

The computer-readable recording medium according to Supplementary note 14,
wherein in the step of determining whether the person to be measured is in the first state, the second state, or the third state,
frequency analysis is performed on the pulse rate, and it is determined that the person to be measured is in the second state or the third state when a peak value exists in a predetermined frequency range.

### Supplementary Note 17

The computer-readable recording medium according to any one of Supplementary notes 14 to 16,
the program further includes an instruction to cause the computer to execute a step of acquiring an acceleration of movement of at least one part of the person to be measured, and
wherein in the step of determining whether the pulse is normal,
the pulse rate is corrected by the acceleration, and the pulse of the person to be measured is determined by the corrected pulse rate.

### Supplementary Note 18

The computer-readable recording medium according to any one of Supplementary notes 14 to 17,
wherein in the step of determining whether the pulse is normal,
frequency analysis is performed on the pulse rate, and it is determined that the pulse of the person to be measured is bradycardia when a frequency range of a first threshold value or less exists, and is determined that the pulse of the person to be measured is tachycardia when a frequency range of a second threshold value or more exists.

### Supplementary Note 19

The computer-readable recording medium according to any one of Supplementary notes 14 to 18,
wherein the pulse rate of the person to be measured is determined from a pulse wave signal of the person to be measured.

Although the invention has been described above with reference to the example embodiment, the invention is not limited to the above example embodiment. Various changes that can be understood by those skilled in the art can be made within the scope of the invention in terms of the structure and details of the invention.

This application claims priority on the basis of Japanese Patent Application No. 2019-53758 filed on Mar. 20, 2019, the entire disclosure of which is incorporated herein by reference.

### Industrial Applicability

As described above, according to the present invention, it is possible to accurately perform the health management of the person to be measured without requiring the personal information such as whether the person to be measured wears the pacemaker and whether the person to be measured suffers from arrhythmia.

### Reference Signs List

1 Pulse determination apparatus

2 Stress determination apparatus

3 Wearable terminal

11 Pulse rate determination unit

12 State determination unit

13 Pulse determination unit

14 Acceleration acquisition unit

21 Data acquisition unit

22 Biological signal storage unit

23 Noise determination unit

24 Employed modality signal determination unit

25 Employed modality signal output unit

26 Stress determination unit.

What is claimed is:

1. A pulse determination apparatus that determines a pulse of a person to be measured, comprising:
a pulse rate determination unit configured to determine a pulse rate of the person to be measured;
a state determination unit configured to determine whether the person to be measured is in a first state in which a pacemaker attached to the person to be measured is operating, a second state in which the pacemaker attached to the person to be measured is not operating, or a third state in which the person to be measured does not wear the pacemaker, based on variation of the pulse rate; and
a pulse determination unit configured to determine whether the pulse of the person to be measured is normal, wherein
the pulse determination unit does not make a determination, when the state determination unit determines that the person to be measured is in the first state, and
the pulse determination unit makes the determination based on the pulse rate, when the state determination unit determines that the person to be measured is in the second state or the third state.

2. The pulse determination apparatus according to claim 1,
wherein the state determination unit determines that the person to be measured is in the first state when the pulse rate is constant, or when the pulse rate increases or decreases at a constant rate.

3. The pulse determination apparatus according to claim 1,
wherein the state determination moans unit performs frequency analysis on the pulse rate, and determines that the person to be measured is in the second state or the third state when a peak value exists in a predetermined frequency range.

4. The pulse determination apparatus according to claim 1, further comprising
an acceleration acquisition unit for acquiring an acceleration of movement of at least one part of the person to be measured,
wherein the pulse determination unit corrects the pulse rate with the acceleration, and determines the pulse of the person to be measured by the corrected pulse rate.

5. The pulse determination apparatus according to claim 1,
wherein the pulse determination unit performs frequency analysis on the pulse rate, determines that the pulse of the person to be measured is bradycardia when a frequency range of a first threshold value or less exists, and determines that the pulse of the person to be measured is tachycardia when a frequency range of a second threshold value or more exists.

6. The pulse determination apparatus according to claim 1,
wherein the pulse rate determination unit determines the pulse rate of the person to be measured from a pulse wave signal of the person to be measured.

7. The pulse determination apparatus according to claim 1, further comprising:
a stress determination unit configured to determine the stress of the person to be measured,
wherein the stress determination unit determines the stress of the person to be measured by using the pulse rate, when the pulse determination unit determines that the pulse of the person to be measured is normal, and
the stress determination unit determines the stress of the person to be measured by using the biological information other than the pulse rate, when the pulse determination unit determines that the pulse of the person to be measured is not normal, or when the state determination unit determines that the person to be measured is in the first state.

8. A pulse determination method for determining a pulse of a person to be measured, comprising:
determining a pulse rate of the person to be measured;
determining whether the person to be measured is in a first state in which a pacemaker attached to the person to be measured is operating, a second state in which the pacemaker attached to the person to be measured is not operating, or a third state in which the person to be measured does not wear the pacemaker, based on variation of the pulse rate; and
determining whether the pulse of the person to be measured is normal,
wherein when determining whether the pulse is normal,
a determination is not made, when the person to be measured is determined to be in the first state, and
the determination is made based on the pulse rate, when the person to be measured is determined to be in the second state or the third state.

9. The pulse determination method according to claim 8,
wherein when determining whether the person to be measured is in the first state, the second state, or the third state,
it is determined that the person to be measured is in the first state when the pulse rate is constant, or when the pulse rate increases or decreases at a constant rate.

10. The pulse determination method according to claim 8,
wherein when determining whether the person to be measured is in the first state, the second state, or the third state,
frequency analysis is performed on the pulse rate, and it is determined that the person to be measured is in the second state or the third state when a peak value exists in a predetermined frequency range.

11. The pulse determination method according to claim 8, further comprising
acquiring an acceleration of movement of at least one part of the person to be measured,
wherein when determining whether the pulse is normal,
the pulse rate is corrected by the acceleration, and the pulse of the person to be measured is determined by the corrected pulse rate.

12. The pulse determination method according to claim 8
wherein when determining whether the pulse is normal,
frequency analysis is performed on the pulse rate, and it is determined that the pulse of the person to be measured is bradycardia when a frequency range of a first threshold value or less exists, and is determined that the pulse of the person to be measured is tachycardia when a frequency range of a second threshold value or more exists.

13. The pulse determination method according to claim 8
wherein when determining the pulse rate
the pulse rate of the person to be measured is determined from a pulse wave signal of the person to be measured.

14. A non-transitory computer-readable recording medium that records a program that causes a computer to determine a pulse of a person to be measured,
wherein the computer-readable recording medium records a program including instructions that cause the computer to perform:
determining whether the person to be measured is in a first state in which a pacemaker attached to the person to be measured is operating, a second state in which the pacemaker attached to the person to be measured is not operating, or a third state in which the person to be measured does not wear the pacemaker, based on determined variation of a pulse rate of the person to be measured; and
determining whether the pulse of the person to be measured is normal,
wherein when determining whether the pulse is normal,
a determination is not made, when the person to be measured is determined to be in the first state, and
the determination is made based on the pulse rate, when the person to be measured is determined to be in the second state or the third state.

15. The non-transitory computer-readable recording medium according to claim 14,
wherein when determining whether the person to be measured is in the first state, the second state, or the third state,
it is determined that the person to be measured is in the first state when the pulse rate is constant, or when the pulse rate increases or decreases at a constant rate.

16. The non-transitory computer-readable recording medium according to claim 14,
wherein when determining whether the person to be measured is in the first state, the second state, or the third state,
frequency analysis is performed on the pulse rate, and it is determined that the person to be measured is in the second state or the third state when a peak value exists in a predetermined frequency range.

17. The non-transitory computer-readable recording medium according to claim 14,
wherein the program further includes an instruction to cause the computer to perform acquiring an acceleration of movement of at least one part of the person to be measured, and
when determining whether the pulse is normal,
the pulse rate is corrected by the acceleration, and the pulse of the person to be measured is determined by the corrected pulse rate.

18. The non-transitory computer-readable recording medium according to claim 14,
wherein when determining whether the pulse is normal,
frequency analysis is performed on the pulse rate, and it is determined that the pulse of the person to be measured is bradycardia when a frequency range of a first threshold value or less exists, and is determined that the pulse of the person to be measured is tachycardia when a frequency range of a second threshold value or more exists.

19. The non-transitory computer-readable recording medium according to claim 14,
wherein the pulse rate of the person to be measured is determined from a pulse wave signal of the person to be measured.
